(19) United States
(12) Patent Application Publication
(10) Pub. No.: US 2022/0264966 A1
(43) Pub. Date: Aug. 25, 2022

(54) WEIGHTED ROBE APPARATUS

(71) Applicant: Gravity Brand Holdings LLC, New York, NY (US)

(72) Inventors: Ronald Chereskin, Brooklyn, NY (US); Michael James Grillo, Brooklyn, NY (US)

(73) Assignee: Gravity Brand Holdings LLC, New York, NY (US)

(21) Appl. No.: 17/678,004

(22) Filed: Feb. 22, 2022

Related U.S. Application Data
(60) Provisional application No. 63/153,746, filed on Feb. 25, 2021.

(51) Int. Cl. A41D 1/02

(52) U.S. Cl. CPC A41D 1/02

(57) ABSTRACT

A robe may include an upper portion, a lower portion, one or more sleeves, or a collar portion. The collar portion may include a hollow interior bound by an interior surface of the collar portion; a first end stitching; and a second end stitching. The robe may further include an opening connecting the hollow exterior to an exterior of the collar portion. The hollow interior may be sized to accept a weighted component. The weighted component may include a plurality of pockets disposed between a first end and a second end of the weighted component.

### CLAIM OF PRIORITY

This application claims priority from U.S. Provisional Patent Application No. 63/153,746, filed on Feb. 25, 2021, the contents of which are incorporated by reference.

### FIELD OF THE DISCLOSURE

The present disclosure relates to exemplary embodiments of a robe, and more particularly, to exemplary embodiments of a robe having a weighted component within the robe.

### INTRODUCTION

In many settings, individuals may encounter difficulty relaxing. This may be due to a variety of factors including restlessness, stress, agitation, or lack of sleep. Achieving relaxation is critical and key to achieving an optimal lifestyle. For example, relaxed individuals may perform better at work, or may sustain healthier relationships with others. As a result, people often turn to devices to aid in achieving relaxation. However, not all devices are suitable or geared towards the person's needs. For example, some people prefer aromatherapy, while others may prefer audio therapy. Another relaxation technique involves placing weighted items on one's person. Such added weight and pressure may make a person feel grounded and relaxed. One issue, however, with these weighted items is that they are often inconvenient to maneuver, move around with, or are otherwise cumbersome.

Therefore, it is desirable to have a wearable weighted clothing item capable of promoting relaxation whilst enabling mobility in the wearer.

### SUMMARY

Other features and advantages will become apparent to those skilled in the art upon review of the following drawings, the detailed description, and the appended claims.

An aspect of the present disclosure may include a weighted robe. The weighted robe may include a robe having an upper portion, a lower portion, one or more sleeves, or a collar portion. The collar portion may include a hollow interior bound by an interior surface of the collar portion; a first end stitching; and a second end stitching. The robe may further include an opening connecting the hollow exterior to an exterior of the collar portion. The hollow interior may be sized to accept a weighted component. The weighted component may include a plurality of pockets disposed between a first end and a second end of the weighted component.

In an embodiment, the weighted robe may further include a zipper portion including a zipper, a first tooth portion, and a second tooth portion. The zipper may be slidably attached to the first tooth portion; the first tooth portion may be attached to a top portion of the weighted component; and the second tooth portion may be attached an inside portion of the collar portion.

In another embodiment, the length of the first tooth portion and the second tooth portion may be approximately equal to the length of the top portion of the weighted component.

In yet another embodiment, the weighted robe may further include a weighted material. Each of the plurality of pockets may configured to accept the weighted material.

In a further embodiment, the weighted material may be glass.

In an embodiment, the glass may be composed, by weight, of 70-75% silicon dioxide; 10-15% sodium oxide; 7-9% calcium oxide; 2-3% aluminum oxide; 1-1.25% magnesium oxide; <1% potassium oxide; and <1% ferric oxide.

In another embodiment, the weighted material may be sand.

In yet another embodiment, the plurality of pockets may include twelve pockets.

In a further embodiment, the distance between the first end stitching, and the second end stitching within the hollow interior may be approximately equal to the distance between the first end and the second end of the weighted component.

In an embodiment, the opening may include a zipper secured to a perimeter of the opening. The zipper may be configured to open or close the opening.

In an aspect of the present disclosure, each pocket within the plurality of pockets may include a front portion, and a rear portion. The front portion and rear portion may include a weighted material. The weighted material in the front portion may be of smaller size than the weighted material in the rear portion.

In another aspect of the present disclosure, the weighted component may further include a rim disposed around a perimeter of the weighted component. The rim may include a wire capable of plastic deformation.

Additional aspects related to this disclosure are set forth, in part, in the description which follows, and, in part, will be obvious from the description, or may be learned by practice of this disclosure.

It is to be understood that both the forgoing and the following descriptions are exemplary and explanatory only and are not intended to limit the claimed disclosure or application thereof in any manner whatsoever.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure. The drawings should, in no way, be construed as limitations on embodiments of the disclosed invention.

FIG. 1 illustrates a front perspective view of a user wearing a weighted robe according to various embodiments of the present disclosure.

FIG. 2 illustrates a front perspective view of a user wearing the weighted robe where a weighted component is shown within the weighted robe according to various embodiments of the present disclosure.

FIG. 3 illustrates a rear perspective view of a user wearing the weighted robe where a weighted component is shown within the weighted robe according to various embodiments of the present disclosure.

FIG. 4 illustrates a side perspective view of a weighted component, which may be separate from the weighted robe according to various embodiments of the present disclosure.

FIG. 5 illustrates a top perspective view of a weighted component, which may be separate from the weighted robe according to various embodiments of the present disclosure.

FIG. 6 illustrates the weighted component being connected to the hollow interior of the collar portion of the weighted robe according to various embodiments of the present disclosure.

### DETAILED DESCRIPTION

For this disclosure, singular words should be construed to include their plural meaning, unless explicitly stated otherwise. Additionally, the term “including” is not limiting. Further, “or” is equivalent to “and/or,” unless explicitly stated otherwise. Although, ranges may be stated as preferred, unless stated explicitly, there may exist embodiments that operate outside of preferred ranges.

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

Aspects of the present disclosure may include a weighted robe 100 having a weighted component 130. The weighted robe 100 may have an opening 120 within a collar portion 110. The opening 120 may open to receive the weighted component 130 and close to secure the weighted component 130.

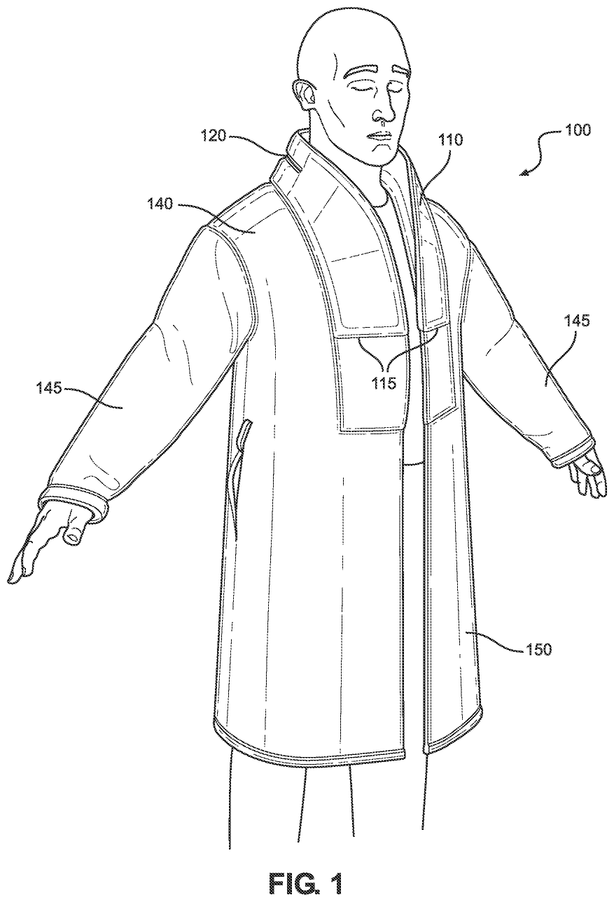

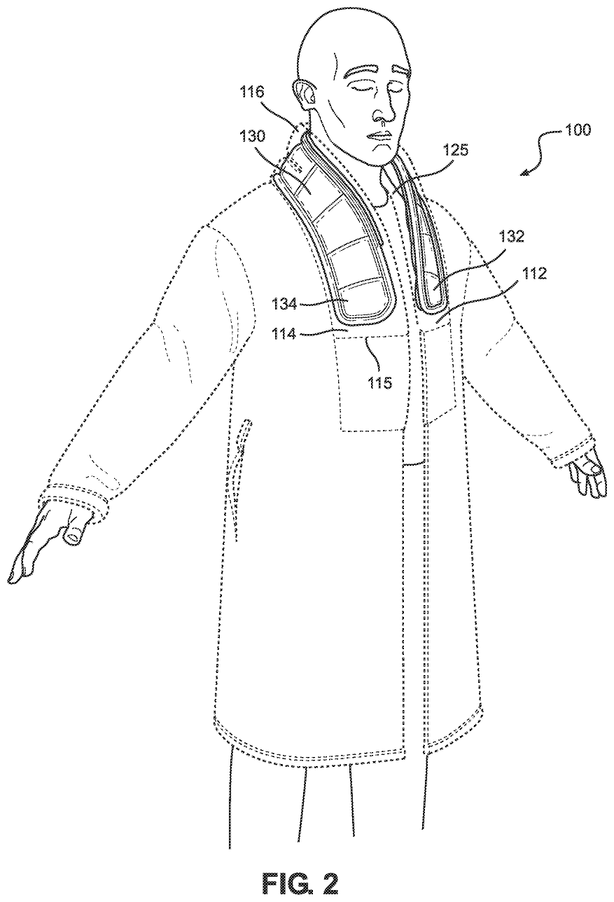

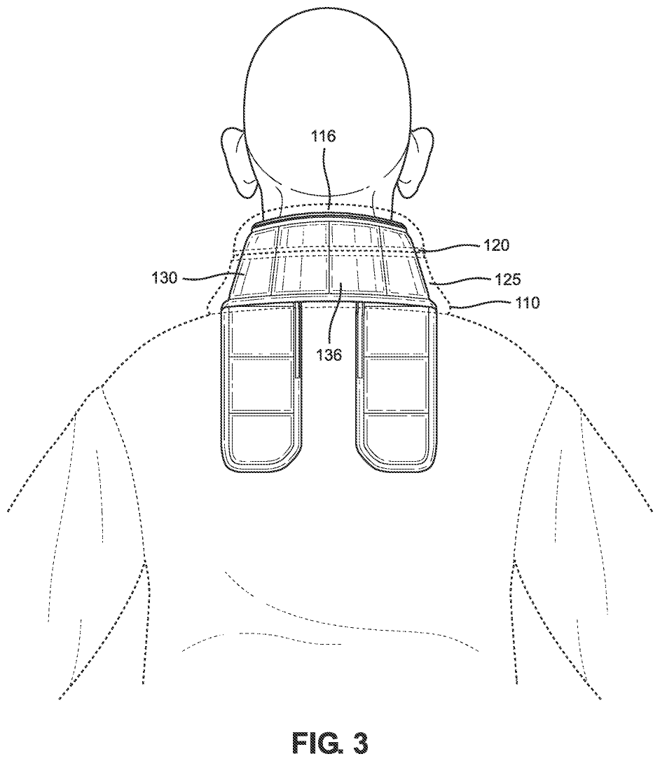

FIG. 1 illustrates a front perspective view of a user wearing a weighted robe 100, and FIGS. 2-3 illustrate a front and rear perspective view, respectively, of a user wearing the weighted robe 100 where a weighted component 130 is shown within the weighted robe 100 according to various embodiments of the present disclosure. The weighted robe 100 may have an upper portion 140 and a lower portion 150, and sleeves 145. However, the weighted robe 100 may have no sleeves 145.

The weighted robe 100 may include a collar portion 110 that extends around a neck area of a user and over a chest portion of the user. However, in alternate embodiments, the collar portion 110, or the weighted component 130 thereof, may be disposed on any suitable portion, surface, or region of the weighted robe 100. For example, in an alternate embodiment, the collar portion 110 and/or the weighted component 130 may extend from the neck over the shoulders and at least partially over the sleeves 145. The collar portion 110 may have a hollow interior 125 that includes a hollow first end 112, a hollow second end 114 and a hollow neck portion 116 located between the hollow first end 112 and hollow second end 114.

The collar portion 110 may have an opening 120 at the neck area of the user through which a weighted component 130 may be provided into the hollow interior 125 of the collar portion 110. The weighted component 130 may be placed such that it fits between the hollow first end 112 and the hollow second end 114, as shown in FIGS. 2-3. The collar portion 110 may be stitched at a bottom of first end 112 and second end 114 so that the weighted component 130 does not extend past the stitched lines 115 on either end, which can keep the weighted component 130 symmetrical around the user's neck. In an embodiment, the distance between the stitched lines 115 of the first and second ends 112/114 is approximately equal to a length of the weighted component 130. The length of the weighted component 130 may be the distance between a first end 132 and second end 134. However, the weighted component 130 may have a length less than that of the distance between the first end 132 and the second end 134. As a non-limiting example, in an embodiment where the weighted component 130 is configured to specifically target the neck and upper back of a wearer, the weighted component 130 may surround the back, left and right sides of a wearer's neck, but may not continue to overlay the wearer's chest.

In an embodiment, the weighted component 130 may be incorporated into the weighted robe 100, such that the weighted component 130 is integral to the weighted robe 100. In such an embodiment, pockets 160 of the weighted component 130 are formed by stitching 162 along the width of the collar portion 110 at various intervals.

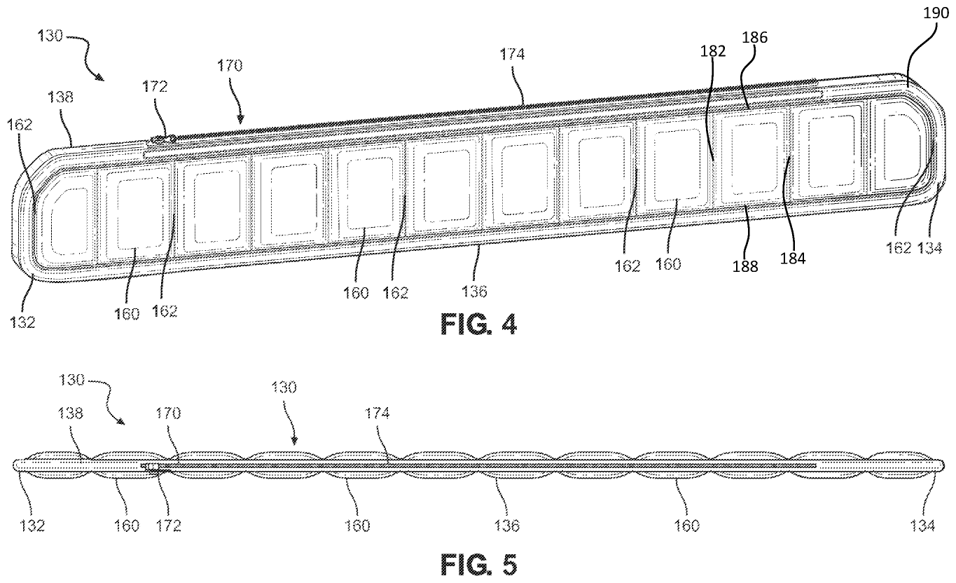

FIGS. 4 and 5 illustrate side and top views, respectively, of the weighted component 130, which may be separate from the weighted robe 100 according to various embodiments of the present disclosure. The weighted component 130 may have a first end 132, a second end 134 opposite the first end 132, and a middle 136 between the first end 132 and second end 134. The weighted component 130 may include a plurality of pockets 160 between the first end 132 and second end 134. The number of pockets 160 may be between one and twenty-five and is not limited to any number. In some embodiments, twelve pockets 160 may be provided in the weighted component 130. Stitching 162 may be provided between the pockets 160 and at the ends to keep the interiors of the pockets separate from other pockets.

Weighted material may be provided in each pocket 160. The stitching 162 may prevent the weighted material from moving around within the weighted component 130 and can keep each pocket 160 the same weight as was designed during the manufacturing process, so that the weighted component 130 does not feel heavier on one end or the middle area. Thus, each stitching 162 may ensure that the predetermined quantity of weighted material remains in each corresponding pocket 160. However, some embodiments may include a gap in the stitching 162 to allow for the movement of weighted material between the pockets 160. In an embodiment, the weighted material is configured in a manner that allows the weighted material to be fixed in place within the weighted component 130. Such weighted material may be fixed via stitching threaded, in part, through a lumen of each individual weighted material (for example, a weighted fabric). Other attachment methods may include adhesive, or any other suitable method known to those skilled in the art.

In some embodiments, the weighted material inside each of the pockets 160 may include a plurality of micro-beads made of, e.g., glass. Other materials may include microfiber beads, sand, steel beads, pebbles, or grains. However, any suitable material known to those skilled in the art may be used. The glass composition may comprise, by weight, a mixture of 70 to 75 percent silicon dioxide, 10 to 15 percent sodium oxide, 7 to 9 percent calcium oxide, 2 to 3 percent aluminum oxide, 1 to 1.25 percent magnesium oxide, less than one percent potassium oxide, and less than one percent ferric oxide. It should be noted that while the glass composition described above is used for illustrative purposes, additional and/or alternative materials may be used to generate other glass compositions that may be used to generate the weighted material, so long as these other glass compositions enable filling of the pockets 160 to achieve a desired weight for the weighted component 130 while maintaining even distribution of the weight material within each pocket 160. In an embodiment, a liquid (e.g., water) may be used to fill the weighted component 130 to achieve a desired weight. In such an embodiment, the weighted component 130 may further include one or more valves. The valves may allow a user to fill or drain the weighted component 130 until a desired weight is achieved. The weighted material may include a variety of other materials and is not limited to any particular weighted material. Different combinations or quantities of weighted materials may be used to achieve a desired weight of the weighted component 130. As a non-limiting example, a particular pocket 160 may include more than one type of weighted material. In such a non-limiting example, a pocket 160 may include a weighted fabric stitched such that it is fixed within the pocket 160 and micro-beads unfixed such that said micro-beans may movably conform to the curvature of the wearer's neck. In another non-limiting example, the type of weighted material may alternate or may otherwise follow a pattern along the weighted component 130. In one embodiment, the three pockets in closest proximity to the first end 132 and the three pockets in closest proximity to the second end 134 may include a first type of weighted material and the remaining middle pockets 160 may include a second type of weighted material.

The weighted material may include a heat retaining material such as rice, corn, dried beans, cherry pits, buckwheat, flaxseed, thermal gel, or birdseed. However, any suitable heat retaining material may be used. In an embodiment, the heat retaining material may be heated in a microwave. In such an embodiment, the materials used to construct the weighted component 130 may include heat and microwave-safe materials such as cotton, wool, linen, or any other suitable material.

In an embodiment, each pocket 160 may be the same dimensions, for example, the same width, height, and/or depth. In a further, embodiment, the shape and dimensions of the pockets 160 may be different along varying portions of the weighted component 130. For example, as shown in FIG. 4, the pocket 160 in closest proximity to the first end 132 and the pocket 160 in closest proximity to the second end 134 may be sized differently than the remaining middle pockets 160. In such an example, said first end 132 and second end 134 pockets 160 may include rounded edges such that the outer edges of the said pockets 160 follow the contour of the first end 132 and the second end 134.

Each pocket 160 may contain multiple layers within it. For example, each pocket 160 may contain an outer layer and an inner layer. The outer and inner layer may be separated by a fabric layer. In an embodiment, to make the weighted component 130 more comfortable when placed around a neck of a user, the outer layer may contain smaller pieces of weighted material than the pieces of weighted material within the inner layer. In another embodiment, each pocket 160 is divided, by a layer of fabric, into a front portion, which faces the neck of the user when worn, and a rear portion, which faces away from the neck of the user when worn. In such an embodiment, the pieces of weighted material in the front portion of the pocket 160 may be smaller than the pieces of weighted material in the rear portion of the pocket 160.

Referring to FIG. 5, each pocket's 160 volume may be uniform along the weighted component 130. However, in further embodiments, each pocket 160 thickness may be adjusted to increase or decrease the volume of a particular pocket 160. In yet a further embodiment, the weighted component 130 may include two or more rows of pockets 160.

Each pocket 160 may comprise a first edge 182, a second edge 184, a top edge 186, and a bottom edge 188. In an embodiment, the first edge 182 and the second edge 184 may be parallel, the top edge 186 and the bottom edge 188 may be parallel, and the first edge 182 and the second edge 184 may be perpendicular to the top edge 186 and the bottom edge 188. In a further embodiment, the first edge 182 and the second edge 184 may be tapered from the bottom edge 188 to the top edge 186, such that the pocket 160 is trapezoidal. Accordingly, the trapezoidal pockets 160 may more easily adjust to the curvature of a wearer's neck. However, the edges 182-188 may be adjusted to any desired or suitable angles. Similarly, the lengths of any of the edges 182-188 may be adjusted for any desired or suitable length. For example, as shown in FIG. 4, the first edge 182 and the second edge 184 may be longer than the top edge 186 and the bottom edge 188.

The weighted component 130 may include a rim 190 surrounding the perimeter of the weighted component 130. The rim 190 may be enclose the weighted component 130 and may provide additional rigidity and structure to the weighted component 130. In an embodiment, the rim 190 may include a wire which is capable of plastic deformation, such that a user may conform the weighted component 130 to a desired shape. Such wire may vary in thickness or density so as to contribute to the desired weight of the weighted component 130. The rim 190 may be constructed from a highly durable material, such as Kevlar, polyethylene, or other suitable material, to maintain the structural integrity of the weighted component 130.

The weighted component 130 may be inserted to fit inside the hollow interior 125 of the collar portion 110 of the weighted robe 100. In some embodiments, the weighted component 130 may include a zipper portion 170 at a top portion 138 of the weighted component 130, which may connect the weighted component 130 to the weighted robe 100. In some embodiments, the zipper portion 170 may include a zip 172 connected to a first tooth portion 174 which is stitched to a top portion 138 of the weighted component 130, as shown in FIGS. 4-5.

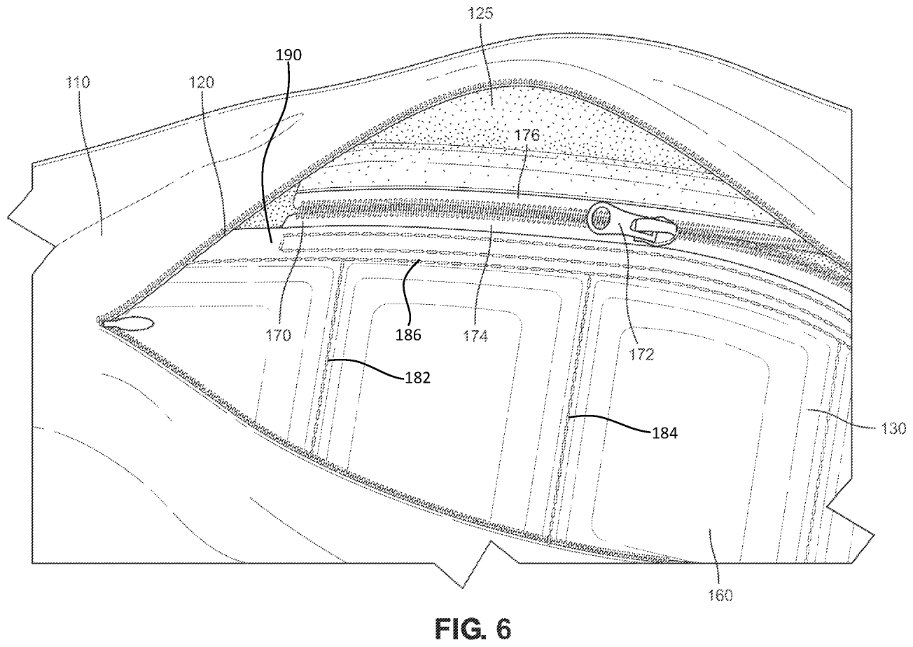

FIG. 6 illustrates the weighted component 130 being connected to the hollow interior 125 of the collar portion 110 of the weighted robe 100 according to various embodiments of the present disclosure. As shown inside the opening 120 (which may include a zipper or other mechanism to seal the hollow interior 125), the weighted component 130 can be placed inside the hollow interior 125. The hollow interior 125 may have a second tooth portion 176 stitched to the hollow interior 125 (i.e., an inside portion of the collar portion 110). The zip 172 can be used to connect the first tooth portion 174 of the weighted component 130 to the second tooth portion 176 of the weighted robe 100 to connect and hold the weighted component inside the hollow interior 125. The first tooth portion 174 may extend along an entire length of a top portion 138 of the weighted component 130 or a portion thereof. Similarly, the second tooth portion 176 may extend along an entire length of the hollow interior 125 or a portion thereof. The first tooth portion 174 and second tooth portion 176 may have a similar length so the zipper portion 170 functions properly. However, in some non-limiting examples, the first tooth portion 174 may be shorter than the second tooth portion 176, or vice versa. The embodiments of the present disclosure are not limited to a zipper portion 170, and various apparatuses may be used to connect the weighted component 130 to the weighted robe 100, such as but not limited to buttons, magnets, ties, hook and loop fasteners or other similar apparatuses. In an embodiment, the weighted component 130 is permanently fixed, via stitching or other methods known to those skilled in the art, within the hollow interior 125 of the collar portion 110 of the weighted robe 100.

Various other considerations may also be addressed in the applications described according to the embodiments of the present disclosure. For example, various materials may be used to construct the elements described in the figures. The weighted robe 100 and the weighted component 130 may be manufactured using various textile materials, such as but not limited to cotton, cashmere, imitation cashmere, fleece, leather, satin, suede, velvet, fustian, corduroy, flannel, tweed, polyester, micro-fiber, and the like. The weighted robe 100 may have various sizes and lengths, and the hollow interior 125 may have various lengths/sizes to accommodate various sizes, lengths and weights of the weighted component 130. The weighted component can have different numbers of pockets, different weights in each pocket. In some embodiments, the pockets 160 may be detachable and then reattached, so that a user or different users may accommodate weighted components having different lengths or weights. In some embodiments, the pockets 160 may be designed to be smaller from a top to bottom portion. For example, further stitching could be provided such that each pocket 160 has a top half and a bottom half (or thirds or quarters), such that further division of the weighted material can be provided so that weight does not feel heavier on a top or bottom portion of each pocket 160 of the weighted component 130. The weighted component 130 may have various lengths, widths and thickness. In some embodiments, the weighted component 130 may have, but is not limited to, a length ranging from between approximately 12 inches to approximately 48 inches, a width ranging from between approximately 2 inches to approximately 8 inches, and a thickness ranging from approximately 0.1 inches to 2 inches.

The words “ranging”, “ranges from”, “ranges between” and other similar notations, are used herein interchangeably and are meant to include the first and second indicated numbers and all the fractional and integral numerals there between. It should be noted that where various embodiments are described by using a given range, the range is given as such merely for convenience and brevity and should not be construed as an inflexible limitation on the scope of the disclosure. Accordingly, the description of a range should be considered to have specifically disclosed all the possible sub-ranges as well as individual numerical values within that range.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, manufacture and methods which, although not explicitly shown or described herein, embody the principles of the disclosure and are thus within the spirit and scope of the disclosure.

What is claimed is:

1. A weighted robe comprising:
a robe including an upper portion, a lower portion, one or more sleeves, and a collar portion,
wherein the collar portion includes:
a hollow interior bound by an interior surface of the collar portion, a first end stitching, and a second end stitching, and
an opening connecting the hollow exterior to an exterior of the collar portion,
wherein the hollow interior is sized to accept a weighted component,
the weighted component including a plurality of pockets disposed between a first end and a second end of the weighted component.

2. The weighted robe of claim 1, further including:
a zipper portion including a zipper, a first tooth portion, and a second tooth portion,
wherein the zipper is slidably attached to the first tooth portion, the first tooth portion is attached to a top portion of the weighted component, and the second tooth portion is attached an inside portion of the collar portion.

3. The weighted robe of claim 2, wherein a length of the first tooth portion and the second tooth portion is approximately equal to a length of the top portion of the weighted component.

4. The weighted robe of claim 1, further including a weighted material,
wherein each of the plurality of pockets are configured to accept the weighted material.

5. The weighted robe of claim 4, wherein the weighted material is glass.

6. The weighted robe of claim 5, wherein the glass is composed, by weight, of:
70-75% silicon dioxide;
10-15% sodium oxide;
7-9% calcium oxide;
2-3% aluminum oxide;
1-1.25% magnesium oxide;
<1% potassium oxide; and
<1% ferric oxide.

7. The weighted robe of claim 4, wherein the weighted material is sand.

8. The weighted robe of claim 1, wherein the plurality of pockets includes twelve pockets.

9. The weighted robe of claim 1, wherein a distance between the first end stitching, and the second end stitching within the hollow interior is approximately equal to a length between the first end and the second end of the weighted component.

10. The weighted robe of claim 1, wherein the opening includes a zipper secured to a perimeter of the opening, the zipper configured to open and close the opening.

11. A weighted robe, comprising:
a robe including an upper portion, a lower portion, one or more sleeves, and a collar portion,
wherein the collar portion includes:
a hollow interior bound by an interior surface of the collar portion, a first end stitching, and a second end stitching, and
an opening connecting the hollow exterior to an exterior of the collar portion,
wherein the hollow interior is sized to accept a weighted component,
the weighted component including a plurality of pockets disposed between a first end and a second end of the weighted component,
wherein each pocket within the plurality of pockets include a front portion, and a rear portion,
wherein the front portion and rear portion include a weighted material, and
wherein the weighted material in the front portion is of smaller size than the weighted material in the rear portion.

12. The weighted robe of claim 11, further including:
a zipper portion including a zipper, a first tooth portion, and a second tooth portion,
wherein the zipper is slidably attached to the first tooth portion, the first tooth portion is attached to a top portion of the weighted component, and the second tooth portion is attached an inside portion of the collar portion.

13. The weighted robe of claim 12, wherein a length of the first tooth portion and the second tooth portion is approximately equal to a length of the top portion of the weighted component.

14. The weighted robe of claim 11, wherein the weighted material is glass.

15. The weighted robe of claim 14, wherein the glass is composed, by weight, of:
70-75% silicon dioxide;
10-15% sodium oxide;
7-9% calcium oxide;
2-3% aluminum oxide;
1-1.25% magnesium oxide;
<1% potassium oxide; and
<1% ferric oxide.

16. The weighted robe of claim 11, wherein the weighted material is sand.

17. The weighted robe of claim 11, wherein the plurality of pockets includes twelve pockets.

18. The weighted robe of claim 11, wherein a distance between the first end stitching, and the second end stitching within the hollow interior is approximately equal to a length between the first end and the second end of the weighted component.

19. The weighted robe of claim 11, wherein the opening includes a zipper secured to a perimeter of the opening, the zipper configured to open and close the opening.

20. A weighted robe, comprising:
a robe including an upper portion, a lower portion, one or more sleeves, and a collar portion,
wherein the collar portion includes:
a hollow interior bound by an interior surface of the collar portion, a first end stitching, and a second end stitching, and
an opening connecting the hollow exterior to an exterior of the collar portion,
wherein the hollow interior is sized to accept a weighted component, the weighted component including:
a plurality of pockets disposed between a first end and a second end of the weighted component, and
a rim disposed around a perimeter of the weighted component,
wherein the rim includes a wire capable of plastic deformation.
